(19) United States
(12) Patent Application Publication
(10) Pub. No.: US 2023/0181076 A1
(43) Pub. Date: Jun. 15, 2023

(54) URINARY FLOW RATE MEASUREMENT DEVICE AND METHOD

(71) Applicant: DOCSHEEN PTY LTD, Edgecliff, New South Wales (AU)

(72) Inventor: Adrian SHEEN, Edgecliff, New South Wales (AU)

(21) Appl. No.: 17/923,663

(22) PCT Filed: May 6, 2021
(86) PCT No.: PCT/AU2021/050420

§ 371 Date: Nov. 7, 2022

(30) Foreign Application Priority Data
May 7, 2020 (AU) 2020901463

(51) Int. Cl. A61B 5/20; A61B 90/00

(52) U.S. Cl. CPC A61B 5/208; A61B 90/39; A61B 2090/3937

(57) ABSTRACT

A urinary flow rate measurement device comprises a vessel having an upper opening configured to receive a urinary input. The device includes a drainage aperture located at or near a base of the vessel, and the device includes a marker located on the wall of the vessel and configured to indicate if the volumetric flow rate of liquid entering the vessel is above or below a predetermined threshold.

### TECHNICAL FIELD

The present disclosure relates to a urinary flow rate measurement device and method. However, it will be appreciated by those skilled in the art that the device may be deployed in other fields of application.

### BACKGROUND OF THE INVENTION

The measurement of urinary volumetric flow rate can be used as an indicator of the presence of several underlying medical conditions. Benign Prostatic Hypertrophy is the most common cause of reduced urine flow, but it can be due to many other conditions which include (but are not limited to) infection, weak bladder muscle and prostatic cancer.

During the lifecycle of a man, the prostate experiences two growth phases. In the second growth phase, commencing around 25 years of age, as the prostate enlarges, the urethra passing through the prostate is narrowed which can cause bladder outlet obstruction leading to thickening of the bladder wall. The bladder may lose the ability to completely empty.

In time there may be a sudden and total inability to pass urine, which is referred to as “acute urinary retention”, which is a medical emergency.

Without suitable treatment, benign prostatic hyperplasia may result in bladder outlet obstruction, reducing urinary flow. As a consequence this can lead to problems which include incomplete voiding, urinary infections, pyelonephritis and bladder calculi. If severe, chronic kidney disease, renal failure and even death can result.

It is estimated that benign prostatic hyperplasia occurs in about 50% of men aged between 50 and 60, and up to 80% of men aged over 80.

Men with benign prostatic hyperplasia may be asymptomatic although symptoms including urinary urgency, a weak urine flow or difficulty commencing urination, and/or nocturnal voiding (nocturia) may be present.

Reduction of volumetric urine flow rate can be a strong indicator of benign prostatic hyperplasia or other medical conditions.

Slowing of urinary flow, regardless of the underlying cause, frequently has a gradual onset over an extended period of time. As such, the person may be unaware of the deterioration in flow rate, until such time as the condition is reasonably advanced.

One test which is available for assessing volumetric urine flow rate is uroflowmetry. This test requires the patient to urinate into a funnel connected to an electronic uroflowmeter. The flow rate is calculated in millilitres per second. Typically a flow rate below 15 millilitres per second may provide an indication of deterioration of flow. Whilst this test can provide useful results, it is generally only undertaken once the person is symptomatic. Furthermore electronic uroflowmeter testing is usually only available in specialist urological practices. As such, the test cannot be readily accessed and would be a significant cost to either public or private health systems.

### Object of the Invention

It is an object of the present invention to overcome or at least ameliorate one or more of the above disadvantages, or at least to provide a useful alternative.

### SUMMARY OF THE INVENTION

In a first aspect, the present invention provides a urinary flow rate measurement device comprising:

a vessel having an upper opening configured to receive a urinary input;

a drainage aperture located at or near a base of the vessel, and

a marker located on the wall of the vessel and configured to indicate if the volumetric flow rate of liquid entering the vessel is above or below a predetermined threshold.

In the preferred embodiment, the predetermined threshold is about 15 millilitres per second.

The urinary marker is preferably defined by a step or band formed on an inner wall of the vessel.

The marker preferably includes text or a region of different colour, such as green or identified by altered texture embedded in the design.

The urinary flow rate measurement device further preferably comprises a urinary stream target locate on a wall of the vessel.

The urinary flow rate measurement device further preferably comprises an arcuate notch formed in the upper opening of the vessel, the notch being located on a generally opposing side of the vessel relative to the target.

In a second aspect, the present invention provides a method of assessing a person's volumetric urinary flow rate, the method including the steps of:

encouraging the person to urinate into an upper opening of a vessel, the vessel having a drainage aperture located at or near a base of the vessel, and a marker located on a wall of the vessel and configured to indicate if the volumetric flow rate of liquid entering the vessel is above or below a predetermined threshold; and

monitoring the level of urine contained within the vessel to determine if the urine level reaches the height of the marker.

The method further preferably includes the step of observing urinary flow rate with the device on a plurality of separate occasions.

### BRIEF DESCRIPTION OF THE DRAWINGS

A preferred embodiment of the invention will now be described by way of specific example with reference to the accompanying drawings, in which:

FIG. 1 is a perspective view of a urinary flow rate measurement device according to the invention;

FIG. 2 is a top view of the device of FIG. 1;

FIG. 3 is a side view of the device of FIG. 1; and

FIG. 4 is a cross-sectional view of the device taken through plane “A” in FIG. 3.

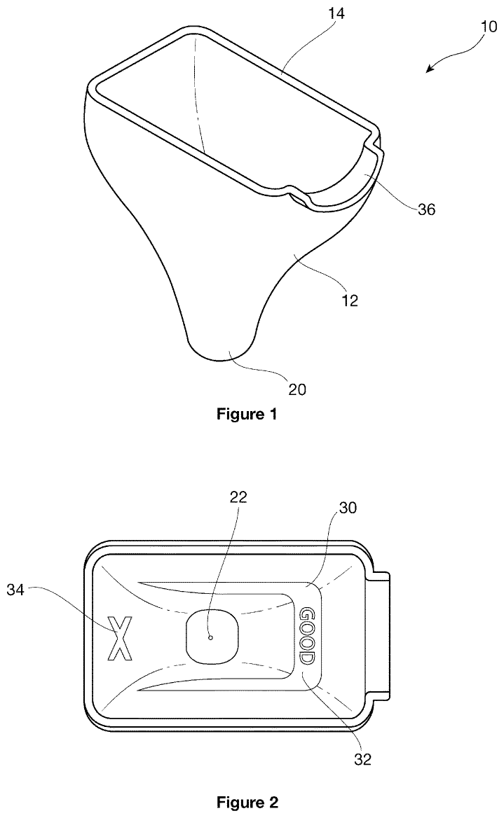

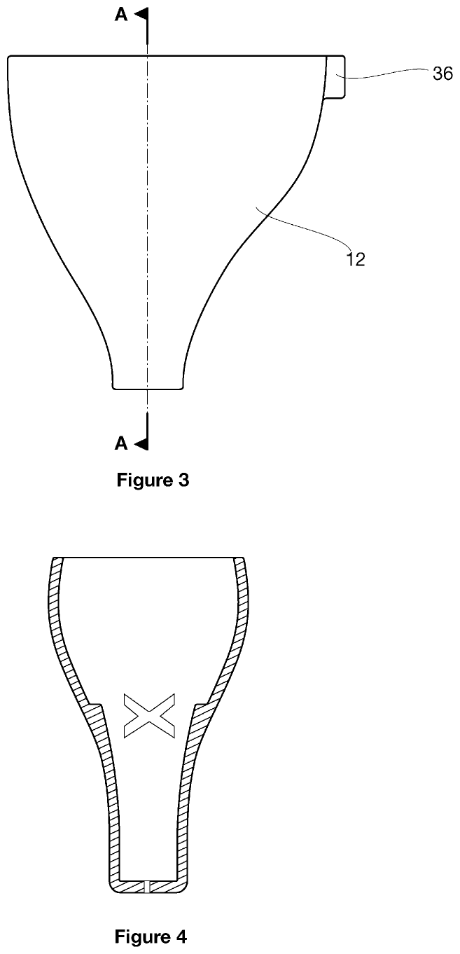

### DETAILED DESCRIPTION OF THE PREFERRED EMBODIMENTS

A urinary flow rate measurement device 10 and a method of using the device 10 is disclosed herein. The device 10 is provided in the form of a vessel 12 having an upper opening 14 defined by the upper most perimeter wall of device 10. The vessel 12 is generally cup-shaped, and funnels such that the largest cross sectional area of the vessel 12 is located at the top, and the vessel 12 reduces in cross-section toward the base 20. As such, the cross-sectional area tapers inwardly, or otherwise reduces from the top toward the bottom. It will be appreciated that the device 10 may be embodied in other shapes.

The vessel 12 is preferably fabricated from a suitable polymer such as polyethylene, polyurethane, polypropylene, polycarbonate, polyamide or other suitable plastics or metals, including stainless steel or aluminium.

The vessel 12 of the preferred embodiment has a maximum volume of about 200 millilitres. However, it will be appreciated that the vessel 12 may be provided in larger or smaller versions.

At or near the base of the vessel 12, the device 10 includes a drainage aperture 22. The drainage aperture is defined by a small hole 22 which is dimensioned to permit urine to drain from the vessel 12. The hole 22 size is preferably between about 3 and 6 mm in diameter, and more preferably between 4 and 5 mm in diameter, and most preferably about 4.3 to 4.6 mm in diameter.

The rate at which urine drains from the device 10, through the drainage aperture 22 is affected by the depth of the urine contained within the vessel 12. The rate at which urine is added to the vessel 12 by the user will vary but when urine is added at 15 millilitres per second in sufficient volume the level of urine will rise to the indicator step 32.

The drainage aperture 22 is sized and located to drain the urine downwardly. The device 10 is intended to be used over a toilet such that the urine passing through the aperture 22 is discharged into the toilet bowl.

In one embodiment not shown in the drawings, the drainage aperture 22 may be in fluid communication with a spout or nozzle, to direct the urine in a desired direction.

If the volumetric flow rate of urine is such that the rate of urine entering the vessel 12 is larger than the rate of urine exiting the drainage aperture 22 then the level of urine within the vessel 12 will gradually rise.

The device 10 includes a marker 30. The marker may be embodied in different forms such as a coloured (for example green) or protruding or embossed band or line or textured region which is located at a fixed level around the wall of the vessel 12. Alternatively, as depicted in the drawings, the marker 30 may be defined by a step 32, such that if the urine level is below the step 32 the volumetric flow rate is insufficient, and if the urine level is at or above the step 32, the volumetric flow rate is sufficient, corresponding to 15 millilitres per second or greater.

The step 32 may include text, such as GOOD, OK, a tick, thumbs up, or some other text, other symbol or textured plastic to indicate to the user that the flow rate is sufficient. The text located on the step 32 can be moulded during fabrication such that it is applied to the internal surface of the vessel 12 during the fabrication stage, obviating the need for colouring and other manufacturing stages.

In a preferred embodiment, the drainage aperture 22 is sized to drain about 15 millilitres per second. This means that if the user's volumetric urine flow rate is at or above 15 millilitres per second, the level of urine within the vessel 12 will gradually rise to the level at or above the marker 30. In contrast, if the user's volumetric urine flow rate is less than 15 millilitres per second, the rate of urine exiting the vessel through the aperture 22 will be such that the level of urine will not reach the marker 30.

The marker 30, which may be embodied as a step 32 or a band or line or textured region, extends horizontally around all of the wall of the vessel 12, or at least a significant portion of the wall of the vessel 12. As such, the marker 30 also provides a visual guide to the user to confirm if the device 10 is being held in a horizontal position, as intended. In practice, the user observes the level of urine rising within the vessel 12, and simultaneously holds the vessel 12 so that the upper surface of the urine is approximately parallel with the marker 30. This assists to improve the accuracy of the measurement, as the device 10 is intended to be operated with the marker 30 extending horizontally.

As depicted in the drawings, the perimeter wall of the vessel 12 includes an arcuate notch 36 configured to be positioned below the underside of the user's penis. The internal wall of the vessel 12 also includes a target 34 in the form of an X or some other visual indicator which directs the user where to aim the flow of urine. The target 34 could be embodied in other forms such as a coloured dot, a bull's eye or some other indicator for the user to direct the flow of urine at. The target 34 provides guidance and assists the user to correctly operate the device 10. Furthermore, the target 34 encourages the user to hold the device 10 in the correct orientation such that the marker 30 is generally horizontal.

The notch 36 is located on a generally opposing side of the vessel 12 relative to the target 34.

The device 10 may include an overflow outlet (not shown). For example, a portion of the upper wall which is located on the opposing side of the device 10 relative to the notch 36 may include a spout to empty urine in the event that the urine level approaches the upper rim of the vessel 12.

The operation of the device 10 will now be described. When a use wishes to evaluate if their volumetric urine flow rate is sufficient, the user urinates into the vessel 12. The user is advised to wait until they have a full bladder before conducting the measurement. In addition, the user is advised to conduct the measurement a number of times, for example ten times, with a view to achieving a sufficiently large volumetric flow rate at least 8 out of ten times, and more preferably 9 out of ten times.

The user places his penis above the notch 36, and urinates whilst directing the urine flow toward the target 34. If the level of urine within the vessel 12 reaches the marker 30, the user notes that the volumetric flow rate was at a rate of about 15 millilitres per sec, or above. In contrast, a maximum urine level below the marker 30 may be an indication of an underlying problem and the user is recommended to seek further medical advice with view to further testing.

In one embodiment, the volume within the vessel 12 up to the marker 30 is about 58 millilitres. In an alternative embodiment, the volume within the vessel 12 up to the marker 30 is smaller, at about 40 millilitres. The smaller volume embodiment may be more suitable in some scenarios, for example for men having smaller maximum bladder volume.

The level of urine contained within the vessel 12 will vary during urination. So long as the urine level reaches or exceeds the vertical height of the marker 30 temporarily, the user can determine that the predetermined flow rate threshold has been achieved.

The device is provided with an aperture 22 which is of a size which results in a urine flow rate of 15 millilitres per second with sufficient volume achieving an equilibrium level at the marker 30.

The cross-sectional area of the vessel 12 is smallest at the bottom, and increases toward the upper opening 14. In order to achieve a urine level above the marker 30, the volumetric flow rate will need to be more than 15 millilitres per second.

Advantageously, the device 10 is intended for home use, and can be used by men in a certain age demographic, for example over 50, so that they can intermittently test their urinary flow rate to identify any reduction of flow at an early stage.

Advantageously, the device 10 can be easily used at home, which enables a user to conduct a number of tests of their urinary flow rate over an extended period of time.

Advantageously the device 10 can be used as a screening tool in conjunction with an annual medical or other such program to detect urinary flow problems at an early stage of development.

In one embodiment (not shown) the device 10 may include a user grip or handle that is ergonomically optimised for comfort during use.

Advantageously, the device 10 makes users aware that they have a slow urine flow which may indicate a medical condition.

Advantageously, the device 10 provides a user with an early warning indication that he has a low urinary flow and should seek further medical advice which may reduce the chances of progressive prostate, bladder and kidney complications.

Advantageously, the device provide an easily accessible, cost effective screening tool which gives an early warning of a reduction in urinary flow rate.

Although the invention has been described with reference to specific examples, it will be appreciated by those skilled in the art that the invention may be embodied in many other forms.

1. A urinary flow rate measurement device comprising:
a vessel having an upper opening configured to receive a urinary input;
a drainage aperture located at or near a base of the vessel, and
a marker located on a wall of the vessel and configured to indicate if the volumetric flow rate of liquid entering the vessel is above or below a predetermined threshold.

2. The urinary flow rate measurement device of claim 1, wherein the predetermined threshold is about 15 millilitres per second.

3. The urinary flow rate measurement device of claim 1, wherein the marker is defined by a step or band formed on an inner wall of the vessel.

4. The urinary flow rate measurement device of claim 1, wherein the marker includes text.

5. The urinary flow rate measurement device of claim 1, wherein the marker includes a region of different colour.

6. The urinary flow rate measurement device of claim 1, wherein the marker includes a region of different texture.

7. The urinary flow rate measurement device of claim 1, further comprising a urinary stream target located on a wall of the vessel.

8. The urinary flow rate measurement device of claim further comprising an arcuate notch formed in the upper opening of the vessel, the arcuate notch being located on a generally opposing side of the vessel relative to the urinary stream target.

9. A method of assessing a person's volumetric urinary flow rate, the method including the steps of:
encouraging the person to urinate into an upper opening of a vessel, the vessel having a drainage aperture located at or near a base of the vessel, and a marker located on a wall of the vessel and configured to indicate if the volumetric flow rate of liquid entering the vessel is above or below a predetermined threshold; and
monitoring the level of urine contained within the vessel to determine if the urine level reaches the height of the marker.

10. The method of claim 9 further including the step of observing urinary flow rate with the device on a plurality of separate occasions.
